(19) United States
(12) Patent Application Publication
(10) Pub. No.: US 2009/0311174 A1
(43) Pub. Date: Dec. 17, 2009

(54) Method For Treating Cancer

(76) Inventor: Barry John Allen, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/996,270

(22) PCT Filed: Jul. 21, 2006
(86) PCT No.: PCT/AU06/01033

§ 371 Date: Aug. 8, 2008

(30) Foreign Application Priority Data
Jul. 21, 2005 (AU) 2005903890

(51) Int. Cl. A61K 51/10; A61P 35/00

(52) U.S. Cl. 424/1.49

(57) ABSTRACT

The present invention relates to methods, kits, compositions and uses thereof for treating cancer. In particular, the present invention relates to methods, kits, compositions and uses thereof for the treatment of metastatic cancer, the treatment of angiogenesis associated with metastatic cancer, inhibiting formation of vasculature associated with metastatic cancer, killing pericytes associated with metastatic cancer and killing cancer cells contiguous with tumour capillaries associated with metastatic cancer, comprising a killing agent conjugated to a protein, and wherein said killing agent conjugated to said protein binds to at least one cell associated with the metastatic cancer.

### TECHNICAL FIELD

The present invention relates to methods, kits, compositions and uses thereof for treating cancer. In particular, the present invention relates to methods, kits, compositions and uses thereof for treating melanoma, and to methods, compounds and kits for treating metastatic cancer.

### BACKGROUND ART

Melanoma is the third most common cancer in Australian women, the fourth most common cancer in Australian men, and the single most common cancer in the age group 15 to 44 years. It typically comprises a very aggressive malignancy that originates in the pigment cells of the skin, otherwise known as melanocytes.

Furthermore, metastatic melanoma continues to be an intractable disease that usually defies every therapeutic modality. The disease is usually exacerbated when malignant cells escape from the primary tumour, enter the bloodstream and ultimately lodge in distant organs, facilitating the development of pre-angiogenic nests of metastatic cancer cells. Angiogenic capillary formation accelerates tumour growth, with rapid development of a clinically significant tumour.

Current therapies for metastatic melanoma include surgery, systemic chemotherapy, regional chemotherapy and immunotherapy. However, once cancer cells have dispersed, therapy is at best palliative only. It is therefore clear that new approaches to the treatment of metastatic melanoma are urgently required.

The present invention is therefore predicated on the surprising and unexpected finding that treatment with an alpha-immunoconjugate (AIC) antibody selectively targets and kills metastatic melanoma.

### SUMMARY OF THE INVENTION

According to a first aspect of the present invention, there is provided a method for the treatment of metastatic cancer, wherein said method comprises administering to a subject a therapeutically effective amount of a killing agent conjugated to a protein, and wherein said killing agent conjugated to said protein binds to at least one cell associated with the metastatic cancer.

According to a second aspect of the present invention, there is provided a method for the treatment of angiogenesis associated with metastatic cancer, wherein said method comprises administering to a subject a therapeutically effective amount of a killing agent conjugated to a protein, and wherein said killing agent conjugated to said protein binds to at least one cell associated with the metastatic cancer.

According to a third aspect of the present invention, there is provided a method for inhibiting formation of vasculature associated with metastatic cancer, wherein said method comprises administering to a subject a therapeutically effective amount of a killing agent conjugated to a protein, and wherein said killing agent conjugated to said protein binds to at least one cell associated with the metastatic cancer.

According to a fourth aspect of the present invention, there is provided a method for killing pericytes associated with metastatic cancer, wherein said method comprises administering to a subject a therapeutically effective amount of a killing agent conjugated to a protein, and wherein said killing agent conjugated to said protein binds to at least one cell associated with the metastatic cancer.

According to a fifth aspect of the present invention, there is provided a method for killing cancer cells contiguous with tumour capillaries associated with metastatic cancer, wherein said method comprises administering to a subject a therapeutically effective amount of a killing agent conjugated to a protein, and wherein said killing agent conjugated to said protein binds to at least one cell associated with the metastatic cancer.

According to a sixth aspect of the present invention, there is provided a method for killing endothelial cells in capillaries associated with metastatic cancer, wherein said method comprises administering to a subject a therapeutically effective amount of a killing agent conjugated to a protein, and wherein said killing agent conjugated to said protein binds to at least one cell associated with the metastatic cancer.

The endothelial cells may be killed by alpha particles emitted from other cells contiguous with tumour capillaries. The other cells may be pericytes or cancer cells.

The endothelial cells may be killed by alpha particles emitted from targeted proliferative endothelial cells.

According to a seventh aspect of the present invention, there is provided a process for preparing a killing agent conjugated to a protein for use in treatment of metastatic cancer, and wherein said killing agent conjugated to said protein binds to at least one cell associated with the metastatic cancer.

According to an eighth aspect of the present invention, there is provided the use of a killing agent and a protein in the preparation of a medicament for the treatment of metastatic cancer, and wherein said killing agent conjugated to said protein binds to at least one cell associated with the metastatic cancer.

The metastatic cancer may be selected from the group comprising liver, ovarian, colorectal, lung, breast, prostate, pancreatic, renal, gastric, cervical, endometrial, oesophageal, brain, head or neck tumours, peritoneal carcinomatosis, sarcoma or melanoma. The metastatic cancer may be melanoma.

The protein may bind to an antigen expressed on the surface of the at least one cell associated with the metastatic cancer.

The protein may be an antibody. The antibody may be a monoclonal antibody. The monoclonal antibody may be a monoclonal anti-MCSP antibody. The monoclonal anti-MCSP antibody may recognize and bind to any epitope of MCSP. The monoclonal anti-MCSP antibody may be a murine anti-MCSP monoclonal antibody. The murine anti-MCSP monoclonal antibody may be selected from the group comprising 9.2.27, 225.28S, 763.4 or TP41.2. The murine anti-MCSP monoclonal antibody may be 9.2.27.

The antibody may be a humanized antibody. The humanized antibody may be a humanized monoclonal antibody. The humanized monoclonal antibody may be a humanized monoclonal anti-MCSP antibody.

The monoclonal antibody may be a monoclonal anti-HMW-MAA antibody. The monoclonal anti-HMW-MAA antibody may recognize and bind to any epitope of HMW-MAA. The monoclonal anti-HMW-MAA antibody may be a murine anti-HMW-MAA monoclonal antibody. The murine anti-HMW-MAA monoclonal antibody may be selected from the group comprising 225.28S, 763.4 or TP41.2.

The antibody may be an anti-urokinase plasminogen activator (uPA) antibody. The anti-urokinase plasminogen activator (uPA) antibody may be a monoclonal anti-urokinase plasminogen activator (uPA) antibody. The monoclonal anti-urokinase plasminogen activator (uPA) antibody may be #394.

The antibody may be an anti-muc1 antibody. The anti-muc1 antibody may be c595.

The antibody may be a breast cancer cell antibody. The breast cancer cell antibody may be selected from the group comprising trastuzumab, rituximab or gemtuzumab ozogamicin.

Additionally or alternatively, the protein may be recombinant.

Additionally or alternatively, the protein may be an inhibitor of plasminogen activator. The inhibitor may be plasminogen activator inhibitor-2 (PAI2). The PAI-2 may recognize and bind to a urokinase plasminogen activator.

The killing agent may comprise a radioisotope. The radioisotope may comprise an alpha-emitting radioisotope. The alpha-emitting radioisotope may be selected from the group comprising Tb-149, At-211, Bi-213, Ac-225, Rn-211, Ra-224, Ra-225, Es-255 or Fm-256. The alpha-emitting radioisotope may be Bi-213.

The at least one cell associated with the metastatic cancer may be selected from the group comprising capillary pericytes, endothelial cells, melanocytes or any other metastatic cancer cell.

According to a ninth aspect of the present invention, there is provided a kit for the treatment of metastatic cancer, wherein said kit comprises a therapeutically effective amount of a killing agent conjugated to a protein, and wherein said killing agent conjugated to said protein binds to at least one cell associated with the metastatic cancer.

According to a tenth aspect of the present invention, there is provided a kit for the treatment of angiogenesis associated with metastatic cancer, wherein said kit comprises a killing agent conjugated to a protein, and wherein said killing agent conjugated to said protein binds to at least one cell associated with the metastatic cancer.

According to an eleventh aspect of the present invention, there is provided a kit for inhibiting formation of vasculature associated with metastatic cancer, wherein said kit comprises administering to a subject a therapeutically effective amount of a killing agent conjugated to a protein, and wherein said killing agent conjugated to said protein binds to at least one cell associated with the metastatic cancer.

According to a twelfth aspect of the present invention, there is provided a kit for killing pericytes associated with metastatic cancer, wherein said kit comprises a killing agent conjugated to a protein, and wherein said killing agent conjugated to said protein binds to at least one cell associated with the metastatic cancer.

According to a thirteenth aspect of the present invention, there is provided a kit for killing cancer cells contiguous with tumour capillaries associated with metastatic cancer, wherein said kit comprises a killing agent conjugated to a protein, and wherein said killing agent conjugated to said protein binds to at least one cell associated with the metastatic cancer.

According to a fourteenth aspect of the present invention, there is provided a kit for killing endothelial cells in capillaries associated with metastatic cancer, wherein said kit comprises administering to a subject a therapeutically effective amount of a killing agent conjugated to a protein, and wherein said killing agent conjugated to said protein binds to at least one cell associated with the metastatic cancer.

The endothelial cells may be killed by alpha particles emitted from other cells contiguous with tumour capillaries. The other cells may be pericytes or cancer cells.

The endothelial cells may be killed by alpha particles emitted from targeted proliferative endothelial cells.

According to a fifteenth aspect of the present invention, there is provided a composition for use in any one or more of the following:

(a) the treatment of metastatic cancer;

(b) the treatment of angiogenesis associated with metastatic cancer;

(c) inhibiting formation of vasculature associated with metastatic cancer;

(d) killing pericytes associated with metastatic cancer; and

(e) killing cancer cells contiguous with tumour capillaries associated with metastatic cancer

wherein said composition comprises a killing agent conjugated to a protein, and wherein said killing agent conjugated to said protein binds to at least one cell associated with the metastatic cancer.

### BRIEF DESCRIPTION OF THE DRAWINGS

The present invention will now be described, by way of example only, with reference to the following drawings.

FIG. 1 shows biological clearance of activity from a tumour in melanoma patients after intralesional injection of an AIC. More than 50% of the AIC is cleared within the half-life of 46 minutes.

FIG. 2 shows uptake of administered activity in vital organs after intralesional injection of an AIC. More than 80% of the activity was eliminated by the end of the monitoring period.

FIG. 3 shows clinical response after TAT in a melanoma patient with intralesional injection of an AIC.

FIG. 4 shows histology of tumour sections as follows: A. untreated tumour showing conventional histology; B. tumour treated with antibody only showing no effect on the tumour; C. tumour treated with TAT using intralesional injection of an AIC and showing debris; D. tumour treated with TAT using intralesional injection of an AIC and showing debris throughout most of the section with an island showing some surviving cells near the blood vessels.

FIG. 5 shows sections as follows: A. control section of unirradiated tumour clear of brown stain; B. section treated with TAT using intralesional injection of an AIC and showing brown stain confirming apoptotic cell death (TUNEL assay); C. cell proliferation marker ki67 showing loss of activity in a portion of the tumour treated with TAT using intralesional injection of an AIC.

FIG. 6 shows serum marker melanoma inhibitory activity protein levels (ng/ml) in melanoma patients at baseline, 2 and 4 weeks post-TAT using intralesional injection of an AIC.

FIG. 7 shows the reduction in melanoma size (original size of large tumours shown by black rings) and number in a melanoma patient's leg after systemic (intravenous) alpha therapy using Bi-213-9.2.27 (targeted anti-vascular alpha therapy (TAVAT)). 20 of 21 tumours disappeared, one tumour reduced from 20 mm to 5 mm. Pathology of the tumour beds show no viable melanoma cells.

### DEFINITIONS

As used herein, the term “comprising” means “including principally, but not necessarily solely”. Furthermore, variations of the word “comprising”, such as “comprise” and “comprises”, have correspondingly varied meanings.

As used herein the terms “treating” and “treatment” refer to any and all uses which remedy a condition or symptoms, prevent the establishment of a condition or disease, or otherwise prevent, hinder, retard, or reverse the progression of a condition or disease or other undesirable symptoms in any way whatsoever.

As used herein the term “effective amount” includes within its meaning a non-toxic but sufficient amount of an agent or compound to provide the desired effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein, the term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies of the present invention may exist in a variety of forms, including for example as a whole antibody, or as an antibody fragment, or other immunologically active fragment thereof, such as complementarity determining regions (CDRs). Similarly, an antibody may exist as an antibody fragment having functional antigen-binding domains, that is, heavy and light chain variable regions. Also, an antibody fragment may exist in a form selected from the group consisting of, but not limited to: Fv, Fab, F(ab)2, scFv (single chain Fv), dAb (single domain antibody), bi-specific antibodies, diabodies and triabodies. Accordingly, the term “antibodies” includes natural antibodies, recombinant antibodies, fragments thereof or synthetic binding agents.

As used herein, the term “AIC” refers to alpha-immunoconjugate. An alpha-immunoconjugate is an alpha-emitting radioisotope conjugated to an antibody or other agents, including but not limited to proteins, for example, the plasminogen activator inhibitor-2 (PAI2) protein.

As used herein, the term “killing agent” refers to a compound, substance or material that is capable of killing, either directly or indirectly, metastatic cancer cells, melanoma cancer cells or cells involved in formation of metastatic vasculature, including but not limited to capillary pericytes. A killing agent may comprise an antibody conjugated to a radioisotope, such as an alpha-immunoconjugate. Similarly, a killing agent may comprise a non-radioactive chemical agent.

As used herein, the term “HMW-MAA” refers to high molecular weight melanoma-associated antigen.

As used herein, the term “TAT” refers to targeted alpha therapy.

As used herein, the term “TAVAT” refers to targeted anti-vascular alpha therapy.

As used herein, the term “RBE” refers to relative biological effectiveness.

As used herein, the term “MCSP” refers to melanoma-associated chondroiten sulfate proteoglycan.

As used herein, the term “HAMA” refers to human anti-mouse antibody.

As used herein, the term “LET” refers to linear energy transfer.

### BEST MODE OF PERFORMING THE INVENTION

The melanoma-associated chondroiten sulfate proteoglycan (MCSP) is a tumour-associated cell surface protein which is a human homologue of the rat protein NG2. It is widely expressed not only by melanoma cells but also in developing and tumour vasculature. In particular, it is expressed on the surface of pericytes which line capillaries. During metastasis, capillary growth can be initiated by metastatic cells circulating in the blood stream and lymphatic system, becoming “leaky” and thereby providing for the formation of tumour-associated vasculature and angiogenesis.

Targeted alpha therapy (TAT) is a therapeutic modality that has the ability to kill isolated cells and cell clusters, and thereby prevent the development of lethal metastatic cancer through localized killing of cancer cells by irradiation with an alpha-emitting radioisotope. When directed to MCSP, the effect of TAT is two-fold: to regress solid tumours by targeted anti-vascular alpha therapy (TAVAT) and then to kill residual isolated cells and cell clusters, thereby stopping the development of lethal metastatic cancer. TAT delivers specifically localized, internal radiotherapy using radionuclides linked to vectors with specific tumour cell-binding properties. From a radiobiological perspective, nuclides that emit alpha particles offer advantages over beta emitting radionuclides because of their short range, high energy, high linear energy transfer and correspondingly high radiobiological effectiveness. Their linear energy transfer (LET) is about 100 times greater than that of beta particles, with consequently higher relative biological effectiveness (RBE). Therefore alpha emitters deposit a much greater fraction of total energy into the targeted cancer cell with fewer nuclear hits required to kill the cancer cell.

The 9.2.27 monoclonal antibody is normally a benign but highly specific antibody that binds MCSP expressed on the surface of melanoma cells and pericytes. However, conjugation of this antibody with ²¹³Bi converted this vector into a highly cytotoxic medicament termed alpha-immunoconjugate (AIC). AIC was effective in selectively targeting and killing melanoma cells, and in destroying “leaky” capillaries involved in angiogenesis. Such capillaries were destroyed by targeting the pericytes and/or capillary contiguous cancer cells that line the capillaries with AIC. In this way, the radioisotope emits alpha radiation that kills capillary endothelial cells, thereby closing down the capillaries, thus depriving metastatic cells of vital nutrients. AIC was therefore demonstrated to possess important anti-neogenic effects.

The inventor has found that 16/16 secondary melanomas were positive to the 9.2.27 monoclonal antibody, and dosimetric calculations, derived from pharmacokinetic data, indicate that AIC was very effective in delivering a high radiation dose to tumours while sparing all normal tissues.

The inventor has therefore demonstrated that intralesional TAT is non-toxic and locally efficacious up to 0.5 mCi. There was no evidence of cytotoxicity for antibody alone and the histology showed almost complete cell kill at 150 μCi with few viable cell clusters. The activity cleared rapidly from organs through the kidneys and bladder. All patients were negative for human anti-mouse antibody (HAMA) response.

A major concern with radiolabeled monoclonal antibodies is the stability of the conjugated system. It is important to prevent the in vivo loss of radio-metal, as the dissociated radiolabel accumulates in bone and/or liver, thus delivering an undesired amount of radiation to non-target tissues/organs. It is significant that continuous accumulation of activity in the kidneys was not observed, as free bismuth accumulates in the kidneys within one hour. The lack of accumulation of renal activity suggests that ²¹³Bi did not significantly disassociate from the antibody i.e. the alpha construct remained a stable compound within the patients.

Intralesional injection of the AIC caused radial diffusion of activity throughout the tumour. As the range of the alpha particles is 80 μm, cell kill can only occur as far as the diffusion of the AIC. The biological clearance of activity from the tumour followed two-component exponential kinetics. The rapid clearing component resulted from vascular clearing of unbound AIC due to a combination of the intratumoral pressure, vascular drainage and leaky tumour capillaries. The slower component indicates that the AIC was specifically and successfully bound to the targeted melanoma cells. Some of the factors affecting tumour retention were the tumour size and vascularity, the injection volume, and the injection procedure.

The average biologically effective intralesional tumour dose per injected activity was 30 (11-98) RBE.cGy/μCi. This wide range reflects the different shapes and sizes of the injected tumours, the needle placement and the vascularisation. This value is some 3000 times that for the kidney (0.01 RBE.cGy/μCi). This striking therapeutic ratio clearly identifies the importance of intralesional therapy.

Accordingly, the present invention provides methods for the treatment of metastatic cancer, for the treatment of angiogenesis associated with metastatic cancer, for inhibiting formation of vasculature associated with metastatic cancer, for killing pericytes associated with metastatic cancer, for killing cancer cells contiguous with tumour capillaries associated with metastatic cancer, and for killing endothelial cells in capillaries associated with metastatic cancer, wherein said method comprises administering to a subject a therapeutically effective amount of a killing agent conjugated to a protein, and wherein said killing agent conjugated to said protein binds to at least one cell associated with the metastatic cancer.

The present invention further provides processes for preparing a killing agent conjugated to a protein for use in treatment of metastatic cancer, and wherein said killing agent conjugated to said protein binds to at least one cell associated with the metastatic cancer.

The present invention moreover provides for the use of a killing agent and a protein in the preparation of a medicament for the treatment of metastatic cancer, and wherein said killing agent conjugated to said protein binds to at least one cell associated with the metastatic cancer.

### Tumours

Those skilled in the art will readily appreciate that the methods and compositions of the present invention find application in the treatment of any tumour type amenable to treatment with AIC. For example the tumours which may be treated using methods of the present invention include but are not limited to liver, ovarian, colorectal, lung, breast, prostate, pancreatic, renal, gastric, cervical, endometrial, oesophageal, brain, head or neck tumours, peritoneal carcinomatosis, sarcoma or melanoma.

### Compositions and Routes of Administration

According to the methods of present invention, compounds and compositions may be administered by any suitable route, either systemically, regionally or locally. The particular route of administration to be used in any given circumstance will depend on a number of factors, including the nature of the tumour to be treated, the severity and extent of the tumour, the required dosage of the particular compounds to be delivered and the potential side-effects of the compounds.

For example, in circumstances where it is required that appropriate concentrations of the desired compounds are delivered directly to the site in the body to be treated, administration may be regional rather than systemic. Regional administration provides the capability of delivering very high local concentrations of the desired compounds to the required site and thus is suitable for achieving the desired therapeutic or preventative effect whilst avoiding exposure of other organs of the body to the compounds and thereby potentially reducing side effects.

By way of example, administration according to embodiments of the invention may be achieved by any standard routes, including intracavitary, intravesical, intramuscular, intraarterial, intravenous, subcutaneous, topical or oral. Intracavitary administration may be intraperitoneal or intrapleural. In particular embodiments, administration may be via intravenous infusion or intraperitoneal administration. Most preferably, administration may be via intravenous infusion.

In general, suitable compositions may be prepared according to methods which are known to those of ordinary skill in the art and may include pharmaceutically acceptable diluents, adjuvants and/or excipients. The diluents, adjuvants and excipients must be “acceptable” in terms of being compatible with the other ingredients of the composition, and not deleterious to the recipient thereof.

Examples of pharmaceutically acceptable diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions. Most preferably, the diluent is saline.

For administration as an injectable solution or suspension, non-toxic parenterally acceptable diluents or carriers can include, Ringer's solution, medium chain triglyceride (MCT), isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

### Dosages

The effective dose level of the administered compound for any particular subject will depend upon a variety of factors including: the type of tumour being treated and the stage of the tumour; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis.

In terms of radioactivity, a therapeutically effective dosage of a composition for intralesional administration to a patient may be in the range of about 10 μCi to 10 mCi, 10 μCi to 5 mCi, 10 μCi to 4 mCi, 10 μCi to 3 mCi, 10 μCi to 2 mCi, 10 μCi to 1000 μCi, 50 μCi to 500 μCi, 50 μCi to 400 μCi, 50 μCi to 300 μCi, 50 μCi to 200 μCi, 100 μCi to 200 μCi, 110 μCi to 190 μCi, 120 μCi to 180 μCi, 130 μCi to 170 μCi, 140 μCi to 160 μCi, 145 μCi to 155 μCi, 146 μCi to 154 μCi, 147 μCi to 153 μCi, 148 μCi to 152 μCi, or 149 μCi to 151 μCi. The therapeutically effective dosage of the composition for intralesional administration to a patient may be 150 μCi.

A therapeutically effective dosage of a composition for systemic administration to a patient may be in the range of about 100 μCi to 100 mCi, 200 μCi to 90 mCi, 300 μCi to 80 mCi, 400 μCi to 70 mCi, 500 μCi to 60 mCi, 600 μCi to 50 mCi, 700 μCi to 40 mCi, 800 μCi to 30 mCi, 900 μCi to 20 mCi, 1000 μCi to 18 mCi, 1.1 mCi to 16 mCi, 1.2 mCi to 14 mCi, 1.3 mCi to 12 mCi, 1.4 mCi to 11 mCi or 1.5 mCi to 10 mCi. The therapeutically effective dosage of the composition for systemic administration to a patient may be in the range of about 1.5 mCi to 50 mCi, or even in a higher range.

The average biologically active effective tumour dose per injected activity for intralesional administration may be in the range of about 1 to 1000 RBE.cGy/μCi, 10 to 100 RBE.cGy/μCi, 10 to 90 RBE.cGy/μCi, 10 to 80 RBE.cGy/μCi, 10 to 70 RBE.cGy/μCi, 10 to 60 RBE.cGy/μCi, 10 to 50 RBE.cGy/μCi, 15 to 45 RBE.cGy/μCi, 20 to 40 RBE.cGy/μCi, 21 to 39 RBE.cGy/μCi, 22 to 38 RBE.cGy/μCi, 23 to 37 RBE.cGy/μCi, 24 to 36 RBE.cGy/μCi, 25 to 35 RBE.cGy/μCi, 26 to 34 RBE.cGy/μCi, 27 to 33 RBE.cGy/μCi, 28 to 32 RBE.cGy/μCi or 29 to 31 RBE.cGy/μCi. The average biologically active effective tumour dose per injected activity may be 30 RBE.cGy/μCi.

The average biologically active effective tumour dose per injected activity for systemic administration may be in the range of about 0.1 to 100 RBE.cGy/mCi, 0.2 to 90 RBE.cGy/mCi, 0.3 to 80 RBE.cGy/mCi, 0.4 to 70 RBE.cGy/mCi, 0.5 to 60 RBE.cGy/mCi, 0.6 to 50 RBE.cGy/mCi, 0.7 to 30 RBE.cGy/mCi, 0.8 to 20 RBE.cGy/mCi, 0.9 to 10 RBE.cGy/mCi, 1.0 to 8 RBE.cGy/mCi, 1.1 to 6 RBE.cGy/mCi, 1.2 to 4 RBE.cGy/mCi, 1.3 to 2 RBE.cGy/mCi, 1.4 to 1.8 RBE.cGy/mCi or 1.5 to 1.7 RBE.cGy/mCi. The average biologically active effective tumour dose per injected activity for systemic administration may be 1.6 RBE.cGy/mCi.

In terms of weight, a therapeutically effective dosage of a composition for administration to a patient is expected to be in the range of about 0.01 mg to about 150 mg per kg body weight per 24 hours; typically, about 0.1 mg to about 150 mg per kg body weight per 24 hours; about 0.1 mg to about 100 mg per kg body weight per 24 hours; about 0.5 mg to about 100 mg per kg body weight per 24 hours; or about 1.0 mg to about 100 mg per kg body weight per 24 hours. More typically, an effective dose range is expected to be in the range of about 5 mg to about 50 mg per kg body weight per 24 hours.

Alternatively, an effective dosage may be up to about 5000 mg/m². Generally, an effective dosage is expected to be in the range of about 10 to about 5000 mg/m², typically about 10 to about 2500 mg/m², about 25 to about 2000 mg/m², about 50 to about 1500 mg/m², about 50 to about 1000 mg/m², or about 75 to about 600 mg/m².

Further, it will be apparent to one of ordinary skill in the art that the optimal quantity and spacing of individual dosages will be determined by the nature and extent of the condition being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per unit time, can be ascertained by those skilled in the art using conventional course of treatment determination tests.

### Kits

The present invention provides kits for use in the treatment of metastatic cancer, the treatment of angiogenesis associated with metastatic cancer, inhibiting formation of vasculature associated with metastatic cancer, killing pericytes associated with metastatic cancer and killing cancer cells contiguous with tumour capillaries associated with metastatic cancer, wherein the kit comprises a killing agent conjugated to a protein, and wherein said killing agent conjugated to said protein binds to at least one cell associated with the metastatic cancer.

The kits of the present invention facilitate the employment of methods of the invention. Typically, kits for carrying out a method of the invention contain all the necessary reagents to carry out the method.

In the context of the present invention, a compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the test sample, a container which contains the antibody(s) used in the assay, containers which contain wash reagents (such as phosphate buffered saline, Tris-buffers, and like), and containers which contain the detection reagent.

Typically, a kit of the present invention will also include instructions for using the kit components to conduct the appropriate methods.

The present invention will now be further described in greater detail by reference to the following specific examples, which should not be construed as in any way limiting the scope of the invention.

### EXAMPLES

### Example 1

### General Methods

### 1.1 Patient Enrolment

Clinical investigators invited eligible melanoma patients to participate in the trial. The patients were informed about the objectives and risks of the study and were required to sign the consent form before study specific screening procedures were performed. This study was approved by the South Eastern Sydney Area Ethics Committee (00/76 Allen) and the NSW Radiation Safety Council and Environmental Protection Authority.

Criteria for entry into the study were resected patients with documented AJCC/UICC (American Joint Committee on Cancer/International Union against Cancer) stage IV melanoma, at least 18 years of age with expected survival of at least 6 months and adequate haematological, renal and liver functions. Patients had to be able to provide informed consent and to have a Karnofsky Performance Status of at least 70%. Patients were excluded if they had active brain metastasis, active infection or serious medical comorbidity, were pregnant or had known allergy to mouse products. Patients were also excluded if their white blood cell (WBC) count was less than 2.0×10⁹/L, platelet count less than 150×10⁹/L, aspartate amino transferase (AST) and alanine amino transferase or serum glutamic oxaloacetic transaminase (ALT) levels more than 3 times the upper limit of normal or serum creatinine level of more than 0.2 mmol/L. Patients who had been treated with chemotherapy, radiotherapy or immunotherapy within 4 weeks were also excluded. Subjects were enrolled as they were identified and confirmed to meet all the criteria for study participation.

A total of 16 patients, most of them referred by Sydney Melanoma Unit for final assessment, satisfied eligibility criteria and were included in the trial. The mean age for men was 75.6 years (range, 56-85 years) and women 71.1 years (range 69-84 years). In 14 of the 16 patients the injected site was located in the leg.

### 1.2 Study Design

The study comprised an open-labeled Phase 1 dose escalation in order to investigate the toxicity and to evaluate the effective dose of the alpha-immunoconjugate, ²¹³Bi-cDTPA-9.2.27, using a cohort dose escalation design. Secondary objectives were to evaluate the evidence for clinical response using a serum marker and immunohistochemistry. Blood was collected at baseline and at 2 and 4 weeks. An adverse event was determined by the presence of grade III/IV non-haematological or grade IV haematological toxicity according to the National Cancer Institute's Common Toxicity Criteria version 2.0 (1998, 2001).

### 1.3 Procedures

The dose of alpha immunoconjugate (AIC) was escalated in steps of 100 μCi, starting from 50 μCi. The activity of the dose was measured in the dose calibrator (Biodex Atomlab 200) just before the intralesional administration of the AIC. Intralesional injection was either intra- or sub-tumoral depending on the size and shape of the tumour to maximize the diffusion of AIC throughout the tumour volume. More than 75% of the patients received intra-tumoral injections.

Radiation monitoring was carried out over 2-3 hours post-injection. The calibrated radiation detector (NaI) was placed against the skin at four major sites of interest at sufficient intervals to establish kinetics. Reference markings were made on the patient's skin to ensure consistent positioning of the probe over these sites of interest. The probe was positioned centrally over the injected tumour to measure activity retention. Kidney measurements were taken with the probe posterior to each kidney and bladder counts from the anterior surface. Patients were kept well hydrated throughout the monitoring period with regular intake of fluids. A urine sample was taken every 30 minutes over the monitoring period. Activity was inadequate for gamma camera measurements at 2 hours post-TAT, being insufficient to delineate organs.

Venous blood samples were collected into heparinized tubes at baseline and at 2 and 4 weeks for analysis of serum biochemistry and to allow assessment of immune response in 2 weeks and correlation with clinical response after the treatment over the period of 4 weeks.

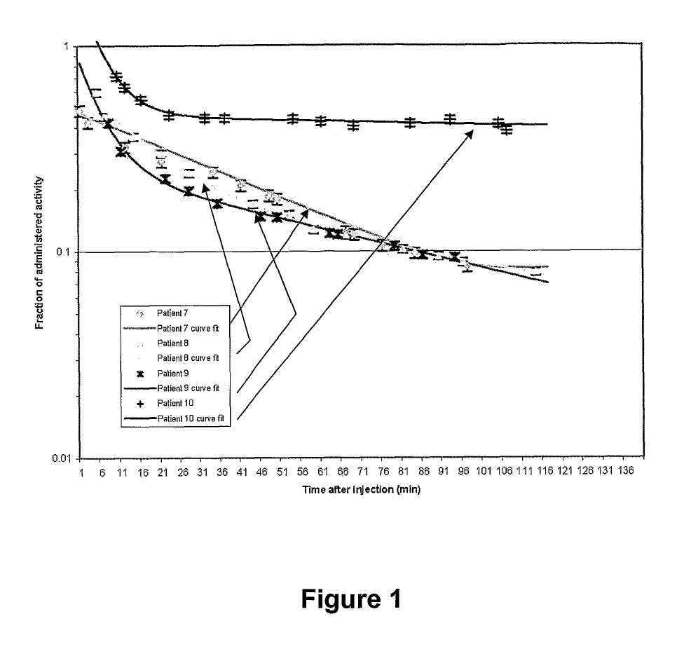

The tumour was photographed at baseline, 2 weeks and 4 weeks to establish any changes in the tumour (FIG. 1). The tumour was excised at 4 weeks. Tumour sections (5 μm in thickness) were prepared and stained for histology and immunohistochemistry. Haematoxylin and Eosin (H & E) staining was used to differentiate tissue cell structure and cell viability. The intact nuclei stained blue whereas the damaged cells did not.

### 1.4 Preparation of Alpha Immunoconjugate (AIC)

Actinium-225 was imported from Department of Energy, United States. The actinium nitrate has purity of 99% ²²⁵Ac with 0.1 mg/mCi of NaNO₃ and less than 0.1 μg/mCi of all other detectable cations. ²¹³Bi is eluted from the Ac-225 generator, and grows back to the initial activity within 2-3 hours. The generator can be eluted within this time period as and when required. Bismuth-213 was eluted from the ²²⁵Ac column using 0.15 M distilled and stabilised hydriodic acid. Bismuth-213 has a 46 minute half-life and emits an 8.36 MeV alpha particle (98%) and a 440 keV gamma ray (17%). The dose calibrator (Biodex Atomlab 200) was calibrated to measure Bismuth-213 activity via its 440 keV gamma emission. The calibration was performed with a calibrated source of Au198, which emits a 412 keV photon, of similar energy to ²¹³Bi.

Monoclonal antibody 9.2.27 was supplied by the Royal Newcastle Hospital from the Scripps Research Institute. The chelator, cyclic anhydride of diethylenetriaminepentacetic acid (cDTPA) was purchased from Aldrich Chemical Company, Australia. Under Good Laboratory Practice, the labeling procedure [1] involved the preparation of chelator cDTPA in chloroform, which was then purified under a stream of nitrogen. Instant Thin Layer Chromatography (ITLC) was performed using Gelman paper (strip size 1×9 cm) and 0.5 M sodium acetate (pH 5.5) as the solvent. The radiolabeled and/or cold conjugate separated from the free radioisotope along the paper, the paper was cut into sections and the gamma emissions from each section counted.

The capability of the AIC to attach to cancer cells and its cell killing ability was ascertained once the labelling efficiency and stability are determined. The stability of AIC was tested with two chelators, namely cyclic anhydride of diethylenetriaminepentacetic acid (cDTPA) and CHX-A″. Both chelating agents yielded high labelling with ²¹³Bi and showed similar (˜20%) leaching with cDTPA and CHX-A″ at 2.5 half lives of the isotope [1]. In the first 30 minutes, the leaching was only 8% and 7% respectively. These reproducible results confirmed the adequate stability of both complexes for ²¹³Bi. Similar results were also found for the DTPA challenge test. Cell binding studies did not show any significant difference between labelled and unlabelled antibodies for either chelator. Thus the conformation of the system remained unchanged with chelation and labelling. cDTPA is commercially available and was therefore the preferred chelator. The required buffers were prepared and sterilized.

### 1.5 Serum Stability Test

Serum was prepared from the freshly drawn human blood specimen. AIC was incubated with serum at 37° C. in a 1:100 ratio for AIC and serum respectively [1]. A sample was drawn out immediately and this zero-time specimen was subjected to ITLC. The contents were subjected to occasional shaking and periodic samples were drawn at 0.5, 1, 1.5 and 2 hours. Each specimen was subjected to ITLC and the relative leaching and stable fractions at each time point were calculated as a function of time.

### 1.6 DTPA Challenge

The AIC was subjected to a DTPA challenge test [1] by incubation with different concentrations of the DTPA to ensure that labelling was specific and stable. In addition, the unchelated antibody was also labelled with the isotope. DTPA (10 μmol) was incubated with the unchelated, labelled antibody and also with the AIC. After a 0.5 and 1 hour incubation at 37° C., all specimens were analysed by ITLC and specific and non-specific labelling of the antibody was determined.

### 1.7 Cell Binding Assay

A series of standardization procedures were carried out for the mAb 9.2.27 [1]. Different concentrations of the antibody were incubated with 0.1 million cells for 15 minutes in flow cytometry tubes. Untreated cells were processed as controls. Cell binding efficiency of the antibody was determined by flow cytometry assay.

### 1.8 Pharmacokinetics by Radiation Monitoring

A collimated single NaI spectrometer (crystal diameter: 60 mm, thickness: 50 mm, collimator diameter: 113 mm, collimator length: 192 mm) with single channel analyser (ESI Nuclear Type 5350 spectrometer) was used to monitor the activity distribution and kinetics in the patients. The probe and spectrometer (ESI Nuclear type 5350) were calibrated to obtain a maximum count rate using a 642-682 keV window for the 661 keV photon of a calibrated caesium-137 source. Counting time was preset to 100 seconds and a 400-480 keV window was set for the 440 kev gamma emission from bismuth-213. A background count was taken prior to the AIC measurements. Data were fitted with two exponential functions (Ae^{-ax}+Be^{-bx}).

Estimates of radiation dose to the injected tumour and to various critical organs were calculated [2] using the conventional Medical Internal Radiation Dose (MIRD) system [3, 4, 5]. The effective dose for alpha radiation for deterministic organ damage uses the recommended value for RBE=5 [6], whereas the Equivalent Dose for stochastic effects or mutagenesis has a radiation weighting factor Wr=20 [7].

### 1.9 Gamma Camera Imaging

A dual headed gamma camera (Prism 2000XP: Marconi, Philips) fitted with a high-energy general purpose collimator was used to obtain planar images of the patients. 180° opposed anterior and posterior images were taken of the lumbar region, consisting of kidneys, liver and bladder. However, activity was too low to obtain useful data.

### 1.10 Human Anti-Mouse Antibody Response (HAMA)

HAMA response was monitored by ELISA assay [8] as the antibody used in the conjugate was of murine origin. Peripheral blood from the patients was collected at day 0 before injection and at 2 and 4 weeks. Two determinations were made and the mean value taken. The presence of antibody was expressed in ng/mL.

### 1.11 Immunoassay

Cell-surface expression of the target antigen was monitored by the alkaline phosphatase anti-alkaline phosphatase (APAAP) and indirect conjugate peroxidase methods on tissues harvested from the patients [9]. APAAP staining was used to detect the presence of the antigenic complex, which bound to the nab 9.2.27. Cells with antigen present on their membrane surface were stained vivid pink.

Tumour sections were also stained with Haematoxylin & Eosin to observe the presence of cell structure. The following tests were also performed to evaluate the evidence of clinical response.

### 1.12 Apoptosis

Apoptosis is a process of programmed cell death characterised by specific morphological changes. These include cell shrinkage, cell fragmentation into small apoptotic bodies. This TUNEL (terminal deoxynucleotide transferase-mediated deoxy uridine nick-end labelling) method uses terminal dideoxynucleotidal transferase (TdT) to incorporate hapten-tagged nucleotides into the 3′ strand breaks that occur in DNA during apoptosis [10]. This is a very sensitive method and is based on detection of DNA strand breaks in early stages of cells undergoing apoptosis. If free 3′ ends in DNA are labelled with biotin-dUTP or DIG-dUTP, the incorporated nucleotides may be detected in a second incubation step with streptavidin. The immunocomplex is easily visible if the streptavidin is conjugated with a reporter molecule (eg fluorescein).

1.13 Cell Proliferation Marker ki67

ki67 is a well known method used to evaluate the proliferation. Studies have shown a strong correlation between proliferation rate and clinical outcome in a variety of tumor types, and measurement of cell proliferative activity is one of several important prognostic markers [11]. This monoclonal antibody reacts with an antigen present in the nucleus of proliferating human cells. Ki-67 expression occurs during the phase of the cell cycle designated as late G1, S, G2 and M. However, the antigen cannot be detected during the G0 phase. This antibody therefore has utility as a marker for cell proliferation.

### 1.14 Melanoma Inhibitory Activity (MIA) Protein

A one step immunoreaction ELISA test was used for the quantification of MIA protein in serum. The “sandwich enzyme immuno-assay” was performed in streptavidin-coated microtitre plates. MIA was simultaneously bound by a biotinylated monoclonal antibody and a peroxidase-conjugated monoclonal antibody that recognizes different epitopes. The complex formed binds to the streptavidin-coated surface of the microtitre plate via the biotinylated antibody with high specificity. The assay time was approx 2 hours. The ELISA test used for the quantification of MIA protein in serum has high specificity. Special additives protect the test system against interfering anti-mouse-antibodies (HAMA) in human sera.

Lower cut-off for positive values (97th percentile) for MIA at 8.8 ng/mL have been reported, based on results for sera from a control group of healthy blood subjects [12].

### Example 2a

### Intralesional Toxicity

Enrolment progressed in a stepwise fashion through the planned dose levels. The patients received intralesional injections of the AIC starting from 50 μCi increasing in steps of 100 μCi. The maximum tolerated dose (MTD) was not reached as an effective intralesional dose was obtained at quite low activities. There were no adverse events. In general, the full blood counts and clinical chemistry did not change from the baseline values. There was no significant red cell abnormality nor change in white blood cells and platelets from baseline. Occasional reactive lymphocytes were seen at higher doses. Slight polychromasia was seen in some patients showing fast platelet turnover. Occasional reactive lymphocytes were seen in some patients. The haemoglobin was in the normal range at all doses. There were no significant changes in sodium, albumin and calcium, urea and creatinine at 4 weeks post-TAT. Potassium did not change and was in the normal range for all the patients. No renal compromise was observed.

All patients reported pain at the injection site, which was graded based on 1-10 scale. For one patient the pain was below 5. However, for an injection into a tumour on the upper forehead, pain was described as 10. For 11/16 patients the pain was at more than 7. However, all the patients described the pain as intense but brief, lasting for 3-4 seconds.

### Example 2b

### Systemic Toxicity

Kinetics data were corrected for radioactive decay and absorbed dose in the tumour and normal tissues was calculated from the measured activities. Dosimetric analysis was based on measured kinematics using a NaI spectrometer located over the injected tumour, bladder and kidneys as well as urine activity measurements. The bi-exponential fit to the clearance of activity showed rather variable fast and slow clearance rates from the tumour. The mean intensities and exponents were found to be:

| | | |
|---|---|---|
| Fast clearance | 0.61 ± 0.09 | 0.10 ± 0.02 |
| Slow clearance | 0.16 ± 0.01 | 0.03 ± 0.03 |
| P value | 0.0002 | 0.003 |

These values are significantly different, indicating that the larger fraction of the AIC is cleared rapidly from the tumour, the smaller fraction being retained by receptors on the melanoma cells. Nevertheless, the intralesional AIC was very effective in delivering a high dose to the tumour while sparing other tissues, the absorbed dose being three orders of magnitude higher in the tumour than the highest dose to any other tissue.

### Example 3

### Retention of Activity in the Tumour

The clearance of activity from the tumour followed two-component exponential kinetics. The biological clearance of activity was characterized by an initial rapid clearance component in which more than 50% of the AIC cleared from the injected tumour within 40 minutes post-TAT (FIG. 1). The second clearing component was much slower indicating a significant portion of the activity remaining at the injection site in the tumour.

### Example 4

### Accumulation of Activity in Bladder and Kidneys

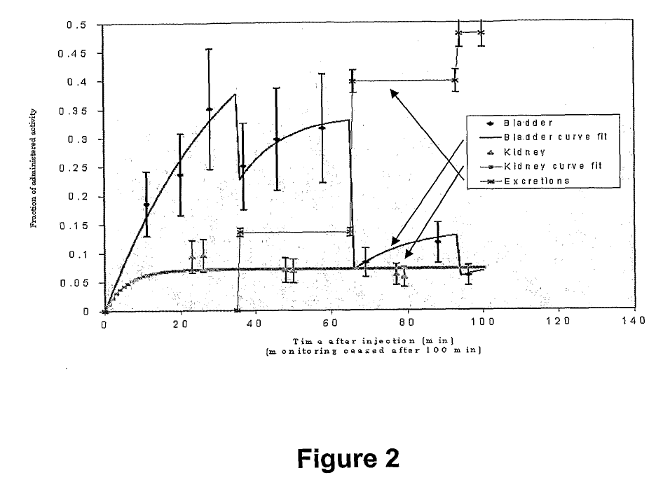

Urine sample counts, corrected for physical decay and sample counter efficiency, showed the amount of activity voided each time the patient urinated. This activity was then compared to the difference in counts measured over the bladder before and after voiding in order to determine the efficiency of bladder measurements. The accumulation of activity in the bladder between voiding changed over time. The uptake rate became progressively slower towards the latter part of the monitoring period (60-100 minutes) until little accumulation in the bladder was observed at all (FIG. 2). The fraction of administered activity remained about 10-20% in almost all patients and more than 80% of the activity was eliminated by the end of the monitoring period.

Urination was the only means of activity excretion. The excreted activity was characterized by a step function, increasing at each urine sampling, the bladder activity being markedly reduced. The activity in the kidneys plateaued at 20 minutes and remained constant at 3-9% of the administered activity throughout the monitoring period. The constant level of activity in the kidneys for most of the monitoring period indicates that the uptake and clearance rates were similar, with no evidence of retention.

### Example 5

### Effective and Equivalent Doses for Intralesional Therapy

Tolerance doses [13] for external beam radiotherapy are defined as the 5% probability for complications arising from the dose being applied to the whole organ, fractionated over 5 days, within 5 years of receiving the dose. In addition, a single dose of 1000 cGy photons to the upper body is well tolerated [14]. These fractionated values can be used as an indicative measure only, as the half-life of Bi-213 is 46 min and 5 day fraction data are not directly applicable.

Cassady [15] generated a dose response curve from available data that showed a threshold for symptomatic radiation nephropathy at 1500 cGy, 5% incidence at 2000 cGy and 95% at 3800 cGy. Renal tolerance (TD 5/5) was measured by Rubin et al [16] to be 2000 cGy for external beam, fractionated photon irradiation of both kidneys [16]. Using the linear-quadratic formula, the equivalent single dose fraction is calculated to be 800 cGy. The renal calculated single fraction tolerance dose is given in Table 1, together with bladder, liver and red bone marrow.

TABLE 1
Estimated RBE and Equivalent Doses in normal organs

| | | | | | Red |
|---|---|---|---|---|---|
| Activity | Kidney | Kidney | Bladder | Liver | marrow |
| μCi | cSv | RBE.cGy | RBE.cGy | RBE.cGy | RBE.cGy |
| 50 | 2.5 | 0.6 | 0.01 | 0.03 | 0.03 |
| 150 | 7.5 | 2 | 0.02 | 0.1 | 0.1 |
| 250 | 12.5 | 3 | 0.005 | 0.2 | 0.2 |
| Single fraction | | 800 | 2600 | 1200 | 1000 |
| tolerance limit | | | | | |
| cGy | | | | | |

Complications are defined in terms of deterministic, or clinically relevant endpoints for each organ, for which RBE=5 for alphas [6] is assumed to be an upper limit. The radiation-weighting factor (W_{R}=20) is used to determine the probability of stochastic events that lead to mutagenesis and carcinogenesis [6]. The unit of effective or RBE dose is RBE.cGy and for the Equivalent Dose for stochastic effects is cSv.

The highest organ dose in this study was that received by the kidneys, for which an average value of 0.01 RBE.cGy/μCi applied for tissue damage, using RBE=5 for alpha radiation. The maximum injected activity in this study was 450 μCi, corresponding to an RBE dose of 4.5 RBE.cGy to the kidney, or 0.06 RBE.cGy/kg for a 70 kg subject. This maximum RBE dose for TAT is only 0.6% of the recommended maximum renal dose.

The effective dose to the bone marrow was estimated to be 0.001 RBE.cGy/μCi, and for 450 μCi, was only 0.45 RBE.cGy or 0.05% of the estimated single fraction tissue tolerance dose of 1000 cGy.

Memorial Sloan Kettering Cancer Center [17] reported that 1 mCi/kg of injected AIC or 70 mCi was safe, with recoverable myeloablation. Our administered maximum activity was 0.6% of this value.

Calculated RBE doses to the injected tumours are given in Table 2 for different administration activities. These doses are some 3000 times greater than those for the organs given in Table 1.

TABLE 2
RBE tumour dose in patients (RBE = 5)

| Administered | Tumour RBE | Equivalent dose per | |
|---|---|---|---|
| Activity A₀ | dose | μCi | RBE dose per μCi |
| μCi | RBE · Gy | Sv/μCi | RBE · Gy/μCi |
| 42-50 | 16-46 | 2.0-5.0 | 0.38-0.98 |
| 144-167 | 7-19 | 0.2-0.7 | 0.04-0.13 |
| 229-262 | 26-44 | 0.6-0.8 | 0.11-0.17 |
| 264-275 | 88-116 | 1.6-2.2 | 0.32-0.44 |

### Example 6

### Evidence of Effective Targeting and Melanoma Cell Kill

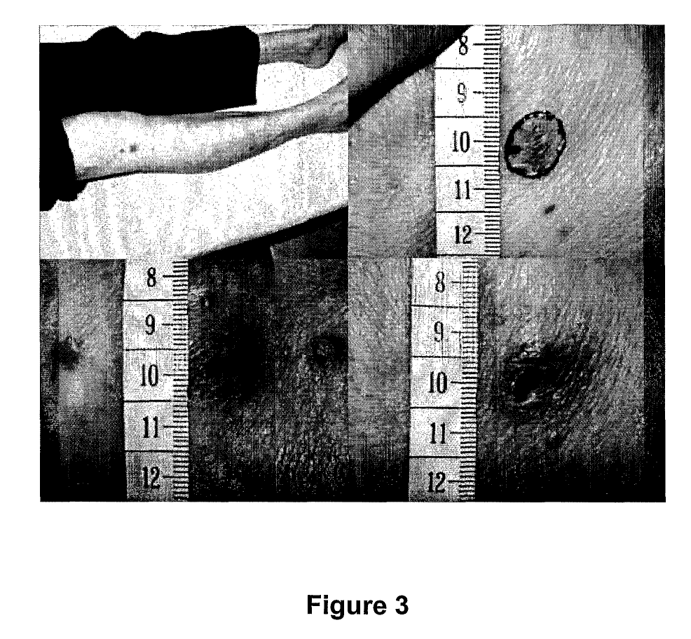

Preliminary evidence of clinical response was obtained by direct observation of the change in skin melanomas (FIG. 3). In general, tumours became larger and softer, as a result of white blood cell invasion. The treated tumour was excised after 4 weeks. The volumes of the excised tumours ranged from 22-1016 mm³.

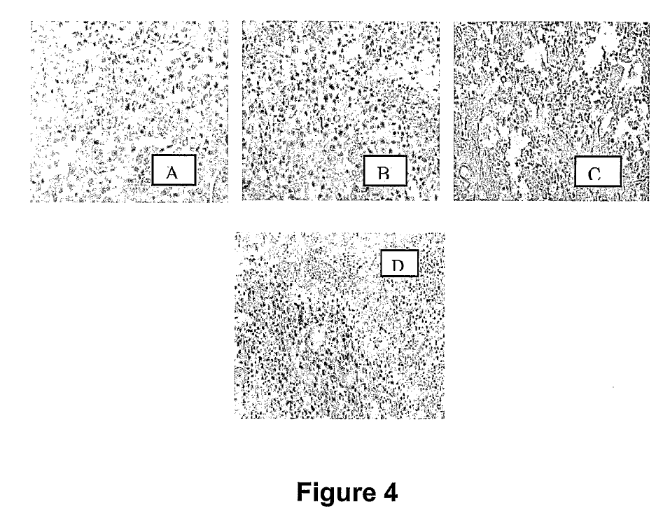

The relative toxic effect of the AIC was tested using 3 lesions in each of 3 patients at a dose level of 250 μCi. Three melanomas of similar size on the skin of each patient were identified; one tumour was left untreated (FIG. 4A), a second tumour was injected with the antibody only (FIG. 4B), and a third tumour was injected with AIC (FIG. 4C). All melanoma cells in the tumour injected with cold antibody survived with similar staining to the untreated tumour, indicating that the antibody alone was not toxic to the melanoma cells, nor did it induce a local HAMA response. However, all AIC treated melanoma cells lost their structure and were replaced by tumour debris, as shown in FIG. 4C. Thus the AIC had targeted and killed the melanoma cells in the injected lesion.

On occasion, not all melanoma cells were killed, and a few surviving cells can generate an island of recurrence if in close proximity to blood vessels. An example is shown in FIG. 4D, where an island of viable melanoma cells (H&E staining) is growing around several capillaries.

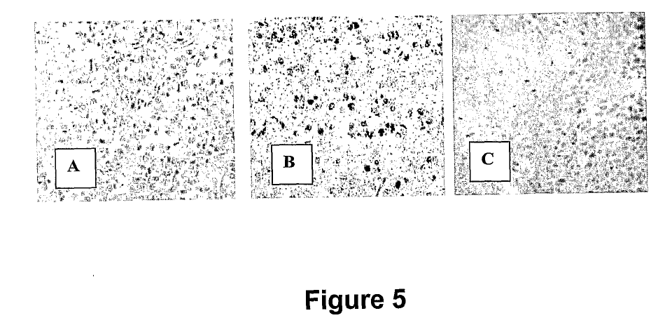

The TUNEL assay showed that the cells died via apoptosis, a process of programmed cell death. The brown stains confirmed the high cell death index (FIG. 5A). Results for cell proliferation ki67 showed a number of cells losing their structure resulting in the reduction in proliferation (FIG. 5B). Thus the immunostaining, apoptosis and ki67 proliferation marker all provide consistent evidence that the AIC targeted and destroyed the melanoma cells.

MIA levels of melanoma patients were compared with healthy subjects. The MIA in the melanoma patients was much higher (15-49 ng/mL) whereas in healthy subjects the maximum MIA was 3.5 ng/mL (P=0.0001).

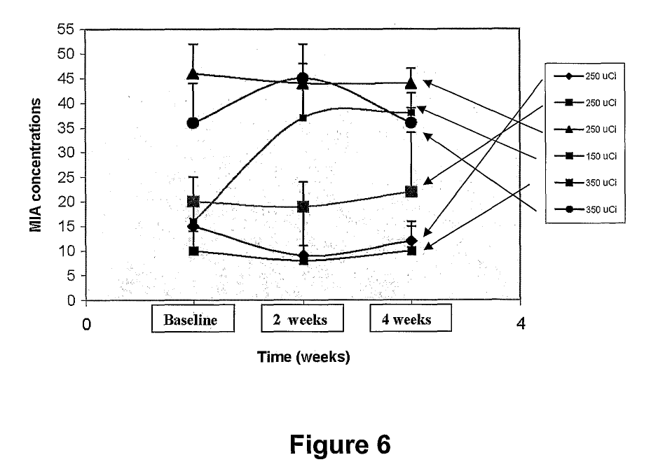

MIA measurements of six melanoma (stage III/IV) patients after treatment were compared at baseline, post-TAT at 2 and 4 weeks (FIG. 6). MIA levels decreased at 2 weeks in four patients, and then increased at 4 weeks and in the other two patients MIA decreased at 4 weeks (P=0.01).

### Example 7

### Clinical Indications

The clinical response was observed by a number of independent methods, including cell-surface expression, apoptosis, Ki67, Melanoma Inhibitory Activity (MIA) protein values and human anti-mouse antibody (HAMA) response.

Cell-surface expression of the target antigen was monitored by the APAAP staining to detect the presence of the antigenic complex, which bound to the mAb 9.2.27. Surviving cells with antigen present on their membrane surface were stained vivid pink whereas the targeted cells did not stain. Results of staining sections from three tumours in three patients showed that cold MAb was completely ineffective, whereas the AIC was highly cytotoxic.

Apoptosis is a process of programmed cell death characterised by specific morphological changes, which include cell shrinkage, cell fragmentation into small apoptotic bodies. The TUNEL assay confirmed a high cell death index as the free ends of DNA were stained brown, as shown in FIG. 5B, compared with the unirradiated tumour section in FIG. 5A.

The Ki67 proliferation marker showed that the melanoma cells were targeted by the AIC, causing a reduction in proliferation, as shown in FIG. 5C.

Values of the Melanoma Inhibitory Activity (MIA) protein for 3 healthy subjects (3.5±0.2 ng/mL) were far below the recommended cut-off values. Enhanced MIA values have been observed in 97% of sera obtained from patients with metastasized malignant melanomas in stage 1V [18], with a significant drop in MIA serum levels after surgery and chemotherapy. Our results confirm a significant decline (P=0.01) in MIA after TAT (FIG. 6) at 4 weeks post-intralesional injections at 200 μCi, suggesting that TAT may have reduced the tumour load. Even a slight decline in MIA values is encouraging at such low doses, as melanoma is a progressive disease and MIA values were expected to increase over 4 weeks whereas we observed that's some MIA values reduced after TAT.

Overall, there is considerable evidence of clinical response indicated by immunostaining, apoptosis, ki67 proliferation marker and serum marker MIA.

HAMA may be produced by human patients as part of an immune response induced by exposure to murine monoclonal antibodies. All patients tested were negative for HAMA response, the HAMA values being below the normal upper limit of 180 ng/mL.

### Example 8

### Systemic Regression of Established Melanomas

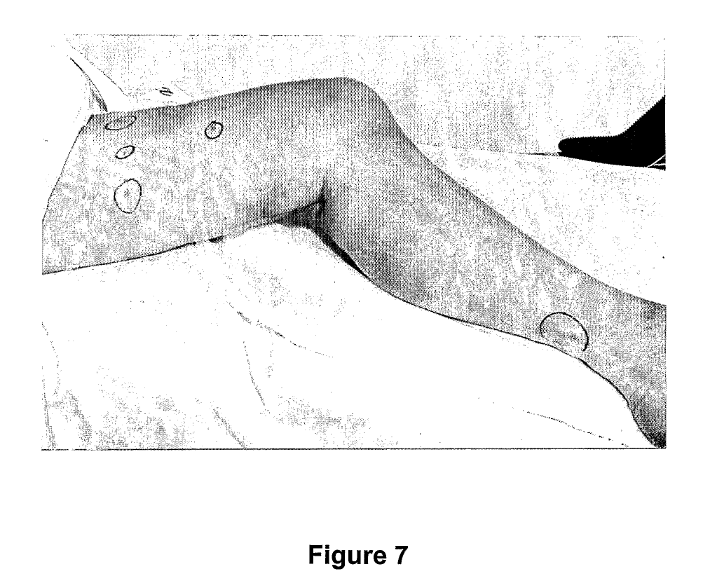

A reduction in melanoma size and number was observed with targeted anti-vascular alpha therapy (TAVAT) in a melanoma patient's leg after a single systemic (intravenous) administration of 1.6 mCi of Bi-213-9.2.27, as shown in FIG. 7. The original size of large tumours is shown by black rings. 20 of 21 tumours disappeared and the one remaining tumour reduced from 20 mm to 5 mm. Pathology of the tumour beds showed no viable melanoma cells. This was an entirely unexpected result, this dose being only a small proportion of the expected maximum tolerance dose, and of the dose used for end stage acute myelogenous leukaemia by the Sloan Kettering Memorial Cancer Center. This result provides a basis for the proposition that not all melanoma cells were killed by TAT during TAT regimens, but rather that neogenic capillaries were closed down by TAVAT, such that the tumours were deprived of nutriments, thereby causing complete regression.

### Example 9

### Clinical Applications of Intralesional TAT

### Example 9.1

### Melanoma Metastasis to Brain

Radiotherapy is the primary treatment for melanoma metastases to the brain. A dose of 3000 cGy is given over 2 weeks, cranial irradiation providing useful palliation to a large majority of patients with brain metastases. There is evidence of improved remission of metastatic melanoma to the brain with accelerated fractionation in some patients. Chemotherapy has a limited role in treating brain metastasis. Many chemotherapy drugs do not cross the blood-brain barrier but can reach malignant tumours in the brain, through a local breakdown in the blood-brain barrier. The intralesional injection of AIC after tumour resection is contemplated as a feasible and efficacious application of TAT with this AIC.

### Example 9.2

### Glioblastoma Multiforme

NG2, being the murine homologue of MCSP, is also expressed by glioblastoma multiforme cells. Prognosis is very poor in that patients live only 6 months to a year after diagnosis. Usually the glioblastoma is seen as a mass lesion involving a focal area. The intralesional injection of AIC after tumour resection is contemplated as a feasible and efficacious application of TAT with this AIC.

### Example 9.3

### Ocular Melanoma

The 5-year overall survival for patients with ocular melanoma is estimated to be 50% to 70% and about 40%-60% of patients develop metastases [19]. The factors related to primary ocular melanoma that influence prognosis include lesion site, cell type and location [19]. The intralesional administration of the AIC to the ocular melanoma is contemplated as a novel approach, which may obviate the need for enucleation and, if followed by systemic TAT, may change the course of the disease.

### REFERENCES

1. Rizvi, S. M. A., Samir, S., Goozee, G., Allen, B. J. 2000. Radioimmunoconjugates for targeted alpha therapy of malignant melanoma. Melanoma Research, 10: 281-290.
2. Tsui W, Phase 1 clinical trial for targeted alpha therapy of metastatic subcutaneous melanoma using Bi-213-cDTPA-9.2.27 MAb. Honours Thesis, UNSW 2001
3. Snyder W, Ford M, Warner G, Watson S. “S” absorbed dose per unit cumulated activity for selected radionuclides and organs. MIRD Pamphlet No 11. Society of Nuclear Medicine, New York, N.Y., 1975.
4. Stabin M. MIRDOSE: the personal computer software for use in internal dose assessment in nuclear medicine. J Nucl Med 1996; 37: 538-46
5. Stabin M, Tagesson M, Thomas S R, et al. Radiation dosimetry in nuclear medicine. Applied Radiation and Isotopes 1999; 50: 73-87
6. Feinendegen L E and J J McClure, 1996 Alpha emitters for medical therapy-workshop of the United States Department of Energy, Meeting report, Denver, Colo., May 30-31.
7. Report of the RBE Committee to the International Commissions on Radiological Units and Measurements, Health Physics 1963: 9; 357
8. Shawler, D. L., Bartholomew, R. M., Smith, L. M., Dillman, R. O. 1985. Human response to multiple injections of murine monoclonal IgG. J. Immunology:135, 1530-1535.
9. Pouly, S., Becher, B., Blain, M., Antel, J P. 1999. Expression of a homologue of rat NG2 on human microglia. Glia 27:259-268.
10. Gorczyca, W., Gong, J., Ardelt, B., Traganos, F., Darzynkiewicz, Z. 1993. The cell cycle related differences in susceptibility of HL-60 cells to apoptosis induced by various antitumor agents. Cancer Research, 53 (13): 3186-3192.
11. Hall, P. A., Levison, D. A. 1990. Assessment of Cell Proliferation in Histologic Material. 1990. J Clinical Pathology, 43(3): 184-92 (review).
12. Bosserhoff, A. K., Kaufmann, M., Kaluza, B., Bartke, I., Zirngibl, H., Hein, R., Stolz, W., Buettner, R. 1997. Melanoma inhibiting activity, a novel serum marker for progression of malignant melanoma. Cancer Research, 57, 3149-53.
13. Emami, B., Lyman, J., Brown, A. Coia, L., Goitein, M., et al. 1991. Tolerance of normal tissue to therapeutic irradiation. International J Radiation Oncol. Biol. Phys., 21:109-122.
14. Mettler, F. A., Upton, A. C., 1995. Medical Effects of Ionizing Radiation, 2nd edn., WB Saunders Company. Philadelphia, US. Chapter 6: pp 214.
15. Cassady, J. R. 1995. Clinical radiation nephropathy. International Journal of Radiation Oncology and Biological Physics: 31(5); 12149-1256.
16. Rubin, P., Casarett, G. W. 1973. Concepts of clinical radiation pathology. In: Dalrymple et al: ed Medical Radiation Biology. 8; 160-189.
17. Jurcic, J. G., Larsen S. M., Sgouros, G. Mcdevitt, M. R., Finn, R. D., Divgi, C. R., Ballangrud, A. M. 2002. Targeted α particle immunotherapy for myeloid leukemia. Blood: 100(4):1233-1239.
18. Bosserhoff, A. K. and Buetner, R. 2002 Expression, function and clinical relevance of MIA (Melanoma Inhibitory Activity) Histology Histopathology: 17:289-300.
19. Feldman, E. D., Pingpank, J. F., Alexander, H. R. 2003. Regional treatment options for patients with ocular melanoma metastatic to the liver. Annals of Surgical Oncology, 11(3): 290-297.

1. A method for the treatment of a vascularized tumour, wherein said method comprises systemically administering to a subject a therapeutically effective amount of an alpha-emitting radioisotope conjugated to an antibody, and wherein said alpha-emitting radioisotope conjugated to said antibody binds to a pericyte associated with the vascularized tumour.

2. The method of claim 1, wherein the treatment of a vascularized tumour comprises treatment of angiogenesis associated with the vascularized tumour.

3. The method of claim 1, wherein the treatment of a vascularized tumour comprises inhibiting formation of vasculature associated with the vascularized tumour.

4. The method of claim 1, wherein the treatment of a vascularized tumour comprises killing pericytes associated with the vascularized tumour.

5. (canceled)

6. The method of claim 1, wherein the treatment of a vascularized tumour comprises killing endothelial cells in capillaries associated with the vascularized tumour.

7-8. (canceled)

9. The method according to claim 1, wherein the vascularized tumour comprises liver, ovarian, colorectal, lung, breast, prostate, pancreatic, renal, gastric, cervical, endometrial, oesophageal, brain, head or neck tumours, peritoneal carcinomatosis, sarcoma or melanoma.

10. The method according to claim 1, wherein the antibody binds to an antigen expressed on the surface of the pericyte associated with the vascularized tumour.

11. The method according to claim 1, wherein the antibody comprises an anti-MCSP antibody.

12-14. (canceled)

15. The method according to claim 1, wherein the alpha-emitting radioisotope comprises Tb-149, At-211, Bi-213, Ac-225, Rn-211, Ra-224, Ra-225, Es-255 or Fm-256.

16-17. (canceled)

18. Use of an alpha-emitting radioisotope and an antibody in the preparation of a medicament for the treatment of a vascularized tumour, and wherein said alpha-emitting radioisotope conjugated to said antibody binds to a pericyte associated with the vascularized tumour, and wherein said medicament is suitable for systemic administration.

19-20. (canceled)

21. A kit for the treatment of a vascularized tumour, wherein said kit comprises a therapeutically effective amount of an alpha-emitting radioisotope conjugated to an antibody, and wherein said alpha-emitting radioisotope conjugated to said antibody binds to a pericyte associated with the vascularized tumour.

22. The kit of claim 21, wherein the treatment of a vascularized tumour comprises treatment of angiogenesis associated with the vascularized tumour.

23. The kit of claim 21, wherein the treatment of a vascularized tumour comprises inhibiting formation of vasculature associated with the vascularized tumour.

24. The kit of claim 21, wherein the treatment of a vascularized tumour comprises killing pericytes associated with the vascularized tumour.

25. (canceled)

26. The kit of claim 21, wherein the treatment of a vascularized tumour comprises killing endothelial cells in capillaries associated with the vascularized tumour.

27-33. (canceled)
